(19) United States
(12) Patent Application Publication
(10) Pub. No.: US 2007/0212417 A1
(43) Pub. Date: Sep. 13, 2007

(54) Compressible resilient granules and formulations prepared therefrom

(76) Inventor: S. Rao Cherukuri, Vienna, VA (US)

Correspondence Address: THORPE NORTH & WESTERN, LLP., 8180 SOUTH 700 EAST, SUITE 350, SANDY, UT, 84070, US

(21) Appl. No.: 11/715,821

(22) Filed: Mar. 7, 2007

Related U.S. Application Data
(60) Provisional application No. 60/780,304, filed on Mar. 7, 2006.

(51) Int. Cl. A61K 31/714; A61K 31/59; A61K 31/525; A61K 31/51; A61K 31/455; A61K 31/4415; A61K 31/355; A61K 31/375; A61K 9/24; A61K 31/12; A61K 31/07

(52) U.S. Cl. 424/471; 424/725; 424/643; 514/52; 514/167; 514/458; 514/251; 514/276; 514/350; 514/356; 514/393; 514/474; 514/725; 514/682

(57) ABSTRACT

Methods and compositions having resilient, self-adhered granules are disclosed and described. In one embodiment, a resilient, self-adhering granule, which includes a combination of a polysaccharide in an amount of about 10 wt % to about 30 wt %; a sugar alcohol in an amount of about 15 wt % to about 35 wt %; and a binder having a viscosity from about 5000 mPa·s to about 250,000 mPa·s in an amount of from about 10 wt % to about 35 wt %, is capable of low-pressure, reversible agglomeration. In other embodiments, an oral dosage form of such granules and methods of administering said forms are provided.

### PRIORITY DATA

This application claims the benefit of U.S. provisional patent application Ser. No. 60/780,304, filed on Mar. 7, 2006, which is incorporated herein by reference.

### FIELD OF THE INVENTION

The present invention relates generally to methods and compositions having resilient granules. Accordingly, the present invention involves the fields of chemistry, biology, and pharmaceuticals.

### BACKGROUND OF THE INVENTION

Pharmaceuticals present in oral dosage forms such as tablets, caplets, capsules, sachets, powders, etc. can have a number of associated problems. When such forms are administered at high doses, the oral dose form can be rather large due to the drug, carrier, and other excipients needed. Such forms are commonly referred to as “horse-pills.” Many patients refuse to take such “horse-pills” and try to cut or crush them into smaller sizes to facilitate easy swallowing. However, in the process, the tablets break and do not provide uniformity in dosing, or because they lack the protective coating in the broken areas, the patient feels the bitter taste of a typical active agent. Neither is an acceptable choice for the patient.

Additionally, such breaking can result in loss of the drug as typical drug compositions easily disintegrate into powders or particles that cannot be easily recovered. As the costs involved in providing drugs can be considerable, such losses can be unacceptable to the patient.

As such, materials and methods for providing oral dosage forms that can be easily modified and avoid the above mentioned problems continued to be sought through on-going research and development efforts.

### SUMMARY OF THE INVENTION

It has been recognized that it would be advantageous to provide an oral dosage form comprised of resilient granules.

Briefly, and in general terms, the invention is directed to methods and compositions having resilient self-adhering granules. In one embodiment, a resilient self adhering granule can include a combination of a polysaccharide in an amount of about 10 wt % to about 30 wt %; a sugar alcohol in an amount of about 15 wt % to about 35 wt %; and a binder having a viscosity from about 5000 mPa·s (milliPascals·s) to about 250,000 mPa·s, in an amount of about 10 wt % to about 35 wt %, that is capable of low-pressure, reversible agglomeration. The granule can further include an active agent or nutritional material. The granule can be configured for immediate release or controlled release formulations. In another embodiment, an oral dosage composition can comprise a plurality of resilient self adhering granules that have agglomerated to form an oral dosage form. The oral dosage form can contain a plurality of active agents in a single granule or active agents individually present in individual granules. The oral dosage form can comprise a tablet that can be broken into two forms with substantially no material loss and can be reformed with substantially no material loss.

In another embodiment, a method of administering an active agent to a subject can comprise providing the active agent in an oral dosage form comprising resilient self-adhering granules, and administering the oral dosage form to the subject's oral cavity such that the majority of the active agent is released in the gastrointestinal tract at a point after the mouth. The active agent can be present in the resilient self-adhering granule or a non-resilient granule. The oral dosage form in some aspects is not a chewing gum and does not use chewing gum base.

There has thus been outlined, rather broadly, the more important features of the invention so that the detailed description thereof that follows may be better understood, and so that the present contribution to the art may be better appreciated. Other features of the present invention will become clearer from the following detailed description of the invention, taken with the accompanying claims, or may be learned by the practice of the invention.

### DETAILED DESCRIPTION OF THE INVENTION

Before the present invention is disclosed and described, it is to be understood that this invention is not limited to the particular structures, process steps, or materials disclosed herein, but is extended to equivalents thereof as would be recognized by those ordinarily skilled in the relevant arts. It should also be understood that terminology employed herein is used for the purpose of describing particular embodiments only and is not intended to be limiting.

It must be noted that, as used in this specification and the appended claims, the singular forms “a,” “an,” and, “the” include plural referents unless the context clearly dictates otherwise. Thus, for example, reference to “a granule” includes one or more of such devices, reference to “an amount of a polysaccharide” includes reference to one or more amounts of polysaccharides, and reference to “the binder” includes reference to one or more binders.

Definitions

In describing and claiming the present invention, the following terminology will be used in accordance with the definitions set forth below.

As used herein, the term “resilient” refers to the ability of a material to retain its general characteristics and/or individuality even though acted upon by an outside force. For example, granules that are resilient may retain many or most of their individual characteristics, such as internal structure, crystallinity, melting point, etc., even when acted upon by mild to moderate external pressures.

As used herein, the phrase “self-adhering” refers to a granule's ability to agglomerate with other like granules or particles under various pressures.

As used herein, the term “free-flowing” refers to the ability to not agglomerate under atmospheric pressure or otherwise to not substantially adhere to objects of a different material.

As used herein, the term “agglomeration” refers to a cluster of like particles in which the particles are held together by surface forces.

As used herein, the phrase, “low-pressure, reversible agglomeration” refers to granules that agglomerate with low pressure, typically about 6500 kilonewtons/m², and that yet retain their individuality to a degree sufficient to allow easy delamination of particles from the agglomerate which are substantially intact with their original individual properties. In some cases, such granules may not substantially rupture or break upon delamination from the agglomerate.

As used herein, the term “sugar alcohol” refers to a hydrogenated form of carbohydrate, whose carbonyl group (aldehyde or ketone, reducing sugar) has been reduced to a primary or secondary hydroxyl group. This term is also commonly known as polyol, polyhydric alcohol, or polyalcohol. Sugar alcohols include, but are not limited to, arabitol, erythritol, hydrogenated starch hydrolysates, isomalt, lactitol, maltitol, mannitol, sorbitol, xylitol, galactitol, inositol, ribitol, dithioerythritol, dithiothreitol, glycerol, and mixtures thereof.

As used herein, the term “carbohydrate” refers to molecules having straight-chain aldehydes or ketones with many hydroxyl groups added, usually one on each carbon atom that is not part of the aldehyde or ketone functional group. Carbohydrates include monosaccharides, disaccharides, oligosaccharides and polysaccharides. Carbohydrates are the most abundant biological molecules, and fill numerous roles in living things, such as the storage and transport of energy (starch, glycogen) and structural components (cellulose in plants, chitin in animals).

Basic carbohydrate units are called monosaccharides, e.g., glucose, galactose, and fructose. The general chemical formula of an unmodified monosaccharide is (C.H₂O)ₙ, where n is any number of three or greater. Monosaccharides can be linked together in almost limitless ways. Two joined monosaccharides are called disaccharides, e.g., sucrose and lactose. Carbohydrates containing between about three to six monosaccharide units are termed oligosaccharides; anything larger than this is a polysaccharide. Polysaccharides, such as starch, glycogen, or cellulose, can reach many thousands of units in length.

As used herein, the term “polysaccharide” refers to polymers made up of many monosaccharides joined together by glycosidic linkages. Generally, they are very large, often branched, molecules. They tend to be amorphous, insoluble in water, and have no sweet taste. Examples of polysaccharides include starch, glycogen, cellulose, and chitin. Polysaccharides have a general formula of Cₙ(H₂O)ₙ₋₁ where n is usually a large number between 200 and 2500. The general formula can also be represented as (C₆H₁₀O₅)ₙ, where n=40-3000. Polysaccharides include, but are not limited to, simple sugars, complex sugars, fibers, starches, pectins, dextrans, dextrins, natural gums, synthetic gums, mucilages, derivatives thereof, components thereof, and mixtures thereof.

As used herein, the term “dextrin” refers to low-molecular-weight carbohydrates produced by the hydrolysis of starch. Dextrins are mixtures of linear α-(1,4)-linked D-glucose polymers. Generally, dextrins are water soluble, white to slightly yellow solids which are optically active. The term dextrin includes maltodextrins.

As used herein, the term “maltodextrin” refers to partially hydrolysated starches. Maltodextrins are defined by the FDA as products having a dextrose equivalent (DE) less than 20. Starch hydrolysates are generally produced by heat, acid, or enzymes. This process breaks down the starch and converts some of the starch to dextrose. With adjustments, this process yields more or less dextrose. Maltodextrins are therefore classified by dextrose equivalence. Dextrose equivalents are a measure of the reducing sugars present calculated as dextrose and expressed as a percentage of the total dry substance. Maltodextrins can have a dextrose equivalent of up to 20. At above 20 DE, the product is then generally classified as corn syrup solids, which are completely soluble and impart significant sweetness.

As used herein, the term “substantially” or “substantial” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking, the nearness of completion will be so as to have the same overall result as if absolute and total completion were obtained. The use of “substantially” is equally applicable when used in a negative connotation to refer to the complete or near complete lack of action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still contain such an item as long as there is no measurable effect thereof.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 micron to about 5 microns” should be interpreted to include not only the explicitly recited values of about 1 micron to about 5 microns, but also include individual values and sub-ranges within the indicated range. Thus, included in this numerical range are individual values such as 2, 3.5, and 4 and sub-ranges such as from 1-3, from 2-4, and from 3-5, etc.

This same principle applies to ranges reciting only one numerical value. Furthermore, such an interpretation should apply regardless of the breadth of the range or the characteristics being described.

The Invention

The present invention provides resilient, self-adhering granules that are capable of low-pressure, reversible agglomeration. For example, when the present granules are pressed between one's thumb and index finger, the granules agglomerate to form a resilient mass, which does not substantially stick to the fingers but rolls freely and can assume any shape desired. Therefore, the granules can be termed free-flowing as well as resilient and compressible. The granules of the present invention are elastic in nature. The plasticity of the granules may arise from their unique internal structure. This is to be contrasted with compressible granules known in the art which are used to form traditional tablets or traditional chewable tablets or orally disintegrating tablets. For example, the granules of the art do not agglomerate but rather typically are destructurized into smaller particles or powder when subjected to light pressure as described in this paragraph. These destructurized smaller particles or powder is an irreversible phenomenon and cannot be re-formed into granules that are substantially of original shape and size unless a very high compression force or some other formulation technique is used.

Because of the unique characteristics of resiliency and agglomeration under light pressure, dosage forms such as tablets and caplets made from present granules are resistant to breakage or chipping. Further, if a patient breaks the tablet or caplet either intentionally to reduce dosage size or by accident, the dosage form can be easily reconstructed to its original shape and mass without any substantial loss of material and with minimal visibly distinct breakage mark. The tablet or caplet can be easily reconstructed to its original shape simply by holding the pieces opposite to each other and by applying light pressure in opposite directions. The pieces simply bond or agglomerate together. This should be contrasted with the art-known dosage forms made from art-known granules wherein the dosage form cannot be reconstructed ordinarily under light pressure.

For example, a traditional art-known tablet with 500 mg or more of active ingredient may become a so-called “horse-pill” when formulated with all needed excipients and can become unwieldy to a patient. The present invention provides several advantages over such a dosage form: a) because of the compactness achieved by virtue of the unique internal structure of the resilient, self-adhering granules, the oral dosage forms produced therefrom do not become so big to be considered “horse-pill”; b) even if the dosage form is considered big for some patients, the breaking of the tablet or caplet is much more easily accomplished compared with traditional tablets; and c) since the granules have a relatively high moisture content, the tablet portions can easily and quickly glide in one's mouth with minimal residence time, which minimizes unpleasant taste of uncoated active drug. To further improve this aspect, the active drug can be taste-masked or coated. While the art-known granules may comprise coated or taste-masked active drug, for the reasons discussed above, because such granules lack the unique internal structure of the presently disclosed granules, the resulting product is not either compact, or difficult to break, or does not provide uniformity of dosing, or does not glide in the mouth as comfortably, or has a longer residence time in the mouth. In addition, because the art-known granules are not resilient, or as resilient as the granules of the present invention, the art-known granules, when presented to a patient in a rapidly disintegrating composition, brings out a powdery gritty mouth-feel to the patient. In contrast, the granules of the present invention create a smooth, pleasant mouth-feel. This is especially advantageous in case of pediatric or geriatric patients or patients who have a compromised saliva production (e.g., xerostemia patients or AIDS patients).

As such, the present invention provides methods and compositions directed to resilient, self-adhering granules. It is noted that when discussing a method of administering an oral dosage form comprising resilient, self-adhering granules or a composition of self-resilient granules, each of these discussions can be considered applicable to each of these embodiments, whether or not they are explicitly discussed in the context of that embodiment. Thus, for example, in discussing the binders present in a resilient, self-adhering granule, those binders can also be used in an oral dosage form comprising the granules or a method of administering such an oral dosage form, and vice versa.

Accordingly, in one embodiment of the present invention, resilient self-adhering granules can include a combination of a polysaccharide in an amount of about 10 wt % to about 30 wt %; a sugar alcohol in an amount of about 15 wt % to about 35 wt %; and a binder having a viscosity from about 5,000 mPa·s to 250,000 mPa·s in an amount of about 10 wt % to about 35 wt %, that is capable of low-pressure, reversible agglomeration.

The sugar alcohols can include, but are not limited to, arabitol, erythritol, hydrogenated starch hydrolysates, isomalt, lactitol, maltitol, mannitol, sorbitol, xylitol, galactitol, inositol, ribitol, dithioerythritol, dithiothreitol, glycerol, derivatives thereof, and mixtures thereof. In one embodiment, the sugar alcohol can be maltitol.

The polysaccharides can include, but are not limited to, simple sugars, complex sugars, fibers, starches, pectins, dextrans, dextrins, natural gums, synthetic gums, mucilages, derivatives thereof, components thereof, and mixtures thereof. In one embodiment, the polysaccharide can be a dextrin. In another embodiment, the dextrin can be maltodextrin.

The resilient, self-adhering granules can be pharmaceutically acceptable. Such pharmaceutically acceptable granules may be inert (i.e., comprise no pharmaceutically active agent) or may comprise one or more pharmaceutically active agents. Pharmaceutical oral dosage forms such as tablets, caplets, capsules, sachets, powders and the like comprising compressible resilient granules of the present invention are also provided. Methods for making and using such granules and dosage forms are also provided.

The resilient, self-adhering granules of the present invention can be compressed into a tablet or a caplet of a desirable shape and size. Such compression can be achieved by using compression forces that are known in the pharmaceutical industry, such as those forces ranging from about 100 to about 4000 lbs/in². However, the present granule can also agglomerate under low-pressure. Generally, such low pressure can be about 6500 kilonewtons/m². Additionally, the granules of the present invention can withstand pressures of up to 40 kilonewtons of compression pressure without losing their resiliency.

The resilient, self-adhering granules of the present invention can have a moisture content ranging from about 0.1% to about 10%. In another aspect, the moisture content can range from about 0.1% to about 6%, from about 0.5% to about 4%, from about 1% to about 4%, from about 1% to about 8%, from about 0.5% to about 7%, from about 3% to about 6%, or from about 0.5% to about 7%. This moisture content is generally considered to be high for a compressible tablet. Thus, in one aspect, the present compressible granules can be characterized as compressible granules of high moisture content.

In another aspect, the present granules can have a water activity less than about 0.6. Alternatively, the water activity may be less than about 0.5, less than about 0.4, less than about 0.3, or less than about 0.2. In some aspects, the water activity may range from about 0.1 to about 0.5, from about 0.1 to about 0.4, from about 0.1 to about 0.3, from about 0.1 to about 0.2, from about 0.2 to about 0.5, or from about 0.15 to about 0.5.

The compressible granules of the present invention can be combined in order form a variety of oral dosage formulations. By way of example without limitation, the granules may be compressed or agglomerated into a tablet or a caplet, and can be combined into semi-solid oral dosage formulations, granule filled capsules, and suspensions. Those of ordinary skill in the art will appreciate the plethora of possible end formulations that can be created by combining together the resilient granules described herein. Regarding tablet and caplets, nearly any shape or size can be employed. The tablets and caplets thus formed can be, for example, round, oval, square, rectangular, cylindrical, oblong, triangular, octagonal, hexagonal, and the like. In addition, the tablets and caplets may be scored to provide dosing flexibility. For example, the tablets can be scored to permit two half dosings, or three one-third dosings (i.e., scored twice), or four one-quarter dosings. Other shapes and scoring configurations are also feasible. Regarding semi-solid oral formulations, such formulations are typically soft and chewable and once broken up in the mouth through mastication are then swallowed in order to administer any included active agent to the subject at a point in the gastrointestinal tract which is past, or beyond, the mouth. It is understood that semi-solid formulations expressly excludes chewing gum, which are by contrast, meant to be chewed for a period of time and then spit out and not swallowed.

The unique internal structure of the present granules can permit dosage forms such as tablets or caplets that can be compact, i.e., have a high drug loading per surface area. Because of this unique characteristic of compactness, the present granules can be used to prepare oral dosage forms such as tablets or caplets that carry a high drug loading and yet permit easy swallowing without creating the fear of choking. The compactness of the present dosage forms may be measured as a function of drug loading versus surface area, i.e., mg/cm². In one aspect, the compactness can be expressed as 0.05, 0.08, 0.1, 0.2, 0.3, 0.4, 0.5, 0.6, or 0.7 in mg/cm². In another aspect, the compactness of present granules may be expressed as ranging from 0.01 to 0.5, 0.01 to 0.1, 0.01 to 0.2, or 0.01 to 0.3, in mg/cm². In another aspect, the compactness may range from 0.05 to 0.1, 0.05 to 0.2, 0.05 to 0.3, 0.05 to 0.4, in mg/cm². In another aspect, the compactness may range from 0.1 to 0.2, 0.1 to 0.3, 0.1 to 0.4, or 0.1 to 0.5, in mg/cm².

In addition to the above characteristics, the compressible granules of the present invention, despite its high moisture content, can be free-flowing and may not substantially stick to metal surfaces such as punches and dies and processing equipment such as granulators, dryers, mixers, coaters, etc. This attribute can permit high volume manufacture of tablets, caplets, capsules, powders, sachets, etc., offering significant commercial advantage. It should be mentioned that it is the prevailing understanding in the industry that high moisture content in granules causes several difficulties in pharmaceutical formulations, and is thus actively discouraged. For example, granules are traditionally dried to have a moisture content of 0.1% or even much less to facilitate free-flow of granules, which is an essential requirement for achieving high-speed high-volume tabletting to meet commercial production demands. Further, it is generally considered that high moisture content is detrimental to acceptable stability of the formulations. However, the present invention has uniquely provided stable, free-flowing compressible granules that are highly desirable both from a consumer point-of-view and from industry point-of-view.

In one aspect, the resilient, self-adhering granules of the present invention can comprise a binder or a mixture of binders wherein at least one of the binders can have a viscosity of at least about 5,000 mPa·s. In some aspects, the viscosity of the binder can be greater than about 6,000 mPa·s. In yet another aspect, the viscosity of the binder can be greater than about 7,000 mPa·s, about 8,000 mPa·s, about 10,000 mPa·s, about 12,000 mPa·s, about 14,000 mPa·s, about 16,000 mPa·s, about 18,000 mPa·s, about 20,000 mPa·s, about 22,000 mPa·s, about 24,000 mPa·s, about 26,000 mPa·s, about 28,000 mPa·s, about 30,000 mPa·s, about 60,000 mPa·s, about 80,000 mPa·s, about 100,000 mPa·s, about 120,000 mPa·s, about 150,000 mPa·s, about 180,000 mPa·s, about 210,000 mPa·s, and about 240,000 mPa·s.

In some aspects, the viscosity of at least one of the binders can range from about 5,000 mPa·s to about 250,000 mPa·s, from about 5,000 mPa·s to about 200,000 mPa·s, from about 5,000 mPa·s to about 180,000 mPa·s, from about 5,000 mPa·s to about 150,000 mPa·s, from about 5,000 mPa·s to about 130,000 mPa·s, from about 5,000 mPa·s to about 100,000 mPa·s, from about 5,000 mPa·s to about 80,000 mPa·s, from about 5,000 mPa·s to about 60,000 mPa·s, from about 5000 mPa·s to about 50,000 mPa·s, from about 5000 mPa·s to about 40,000 mPa·s, from about 5000 mPa·s to about 30,000 mPa·s, from about 5,000 mPa·s to about 25,000 mPa·s, from about 5,000 mPa·s to about 20,000 mPa·s, from about 5000 mPa·s to about 15,000 mPa·s, and from about 5,000 mPa·s to about 10,000 mPa·s.

In some aspects, the viscosity of at least one of the binders can range from about 7,000 mPa·s to about 30,000 mPa·s, from about 7,000 mPa·s to about 25,000 mPa·s, from about 7,000 mPa·s to about 20,000 mPa·s, from about 7,000 mPa·s to about 15,000 mPa·s, from about 7,000 mPa·s to about 13,000 mPa·s, and from about 7,000 mPa·s to about 10,000 mPa·s, from about 7,000 mPa·s to about 250,000 mPa·s, from about 7,000 mPa·s to about 200,000 mPa·s, from about 7,000 mPa·s to about 180,000 mPa·s, from about 7,000 mPa·s to about 150,000 mPa·s, from about 7,000 mPa·s to about 130,000 mPa·s, and from about 7,000 mPa·s to about 100,000 mPa·s, from about 7,000 mPa·s to about 80,000 mPa·s, from about 7,000 mPa·s to about 70,000 mPa·s, from about 7,000 mPa·s to about 60,000 mPa·s, from about 7,000 mPa·s to about 50,000 mPa·s, and from about 7,000 mPa·s to about 40,000 mPa·s.

In some aspects, the viscosity of at least one of the binders can range from about 10,000 mPa·s to about 30,000 mPa·s, from about 10,000 mPa·s to about 25,000 mPa·s, from about 10,000 mPa·s to about 20,000 mPa·s, from about 10,000 mPa·s to about 15,000 mPa·s, and from about 10,000 mPa·s to about 13,000 mPa·s, from about 10,000 mPa·s to about 250,000 mPa·s, from about 10,000 mPa·s to about 200,000 mPa·s, from about 10,000 mPa·s to about 180,000 mPa·s, from about 10,000 mPa·s to about 150,000 mPa·s, from about 10,000 mPa·s to about 120,000 mPa·s, from about 10,000 mPa·s to about 100,000 mPa·s, from about 10,000 mPa·s to about 80,000 mPa·s, from about 10,000 mPa·s to about 70,000 mPa·s, from about 10,000 mPa·s to about 50,000 mPa·s, and from about 10,000 mPa·s to about 40,000 mPa·s.

In some aspects, the viscosity of at least one of the binders can range from about 8,000 mPa·s to about 250,000 mPa·s, from about 8,000 mPa·s to about 200,000 mPa·s, from about 8,000 mPa·s to about 150,000 mPa·s, from about 8,000 mPa·s to about 100,000 mPa·s, from about 8,000 mPa·s to about 80,000 mPa·s, and from about 8,000 mPa·s to about 50,000 mPa·s.

In some aspects, the viscosity of at least one of the binders can range from about 10,000 mPa·s to about 28,000 mPa·s, from about 10,000 mPa·s to about 23,000 mPa·s, from about 10,000 mPa·s to about 18,000 mPa·s, from about 10,000 mPa·s to about 14,000 mPa·s, and from about 10,000 mPa·s to about 13,000 mPa·s.

It should be understood that a mixture of binders may be used in the present invention such that the binders in combination may possess a viscosity as recited in the previous paragraphs. For example, a binder such as Lycasin (maltitol syrup) of a lower viscosity (e.g., 2,000 mPa·s) may be combined in different proportions with another binder of higher viscosity (e.g., Lycasin HBC, maltitol syrup of 13,000 mPa·s or Lycasin HDS maltitol syrup with a viscosity of about 250,000 mPa·s) to provide an overall viscosity for the binder mixture of at least 5,000 mPa·s, or other viscosities recited hereinabove. One of ordinary skill in the art is expected to be quite familiar with the arithmetic calculations necessary to arrive at the amounts of each contributing binder.

It is to be further noted that the viscosities stated herein are those stated in the literature for the particular product as mentioned by the manufacturer of the product or in the reference books such as Handbook of Excipients or other equivalent source. In some cases, the viscosities are measured at a certain temperature, for example at 20° C. or 30° C. One of skill in the art would readily note that viscosity is often a function of temperature over a certain range of temperatures for most products. For example, Lycasin HDS is a maltitol syrup manufactured by Roquette America, Inc. The manufacturer states that Lycasin HDS has a viscosity of about 250,000 mPa·s at 30° C. The viscosity for this product cannot be measured at temperatures below 30° C., without some extraordinary effort. Therefore, this is the viscosity that is being used in this application for this particular product. Since one of skill in the art would appreciate this technical aspect, other products listed in the present application have been similarly characterized as necessary, which one skilled in the art would recognize.

Examples of binders that may be used in the present invention include: syrups such as maltitol syrups of varying viscosities; emulsifiers that can function as binders; fats and waxes that can function as binders; and gums that function as binders. Examples of emulsifiers that function as binders include, but are not limited to, acetylated mono, di, or triglycerides or other esters; or polyethyleneglycol esters. Additionally, plasticizers may also be used. Examples of fats and waxes that function as binders include, but are not limited to, bees wax, carnuba wax, spermaceti, etc. Synthetic waxes include, but are not limited to, mineral oil, paraffin, microcrystalline wax, and polyethylene wax. Examples of gums that function as binders include, but are not limited to, gum Arabica, gum tragacanth, gum acacia, and fiber gums. In one embodiment, the binder can be a maltitol syrup.

The compressible resilient granules of the present invention may be inert, i.e., do not comprise a pharmaceutically active agent, or may comprise one or more pharmaceutically active agents. The active agent may be present homogenously throughout the granule or coated on the surface of the granule. In any case, the granules may be used to prepare oral dosage forms such as tablets, caplets, capsules, sachets, powders and the like. For example, where the compressible resilient granules are inert, the granules may be used to provide sufficient flexibility for other ingredients in the dosage form, including other granules, be they art-known granules that are not resilient or be they resilient granules of this invention, so that such ingredients can be compressed into a tablet or caplet or such ingredients can be compressed into a mass of material that can be filled into a capsule.

In one embodiment, the resilient, self-adhering granules of the invention can be inert and mixed with art-known non-resilient granules which can comprise a pharmaceutically active agent and, for example, coated with a taste-masking coating material or a modified release coating material. These two types of granules (resilient and non-resilient) can be compressed into a dosage form such as a tablet or a caplet where there is no substantial breakage or loss of coating on the non-resilient granules. This is a significant advancement in the pharmaceutical arts because the problem of breakage of the coating of coated granules during compression is a well-known problem in the art. For example, enteric coated granules are not generally compressed for the fear that the enteric coating will break under the compression forces generally employed in pharmaceutical tabletting, and thus, cause premature leakage of the active drug in the stomach, before the drug reaches its intended target, namely, the intestines. Similarly, taste-masked granules are generally known to rupture during compression, causing the drug to dissolve in the mouth while swallowing and thus creating a bitter unpleasant taste for the patient. As such, the present invention provides oral dosage compositions that provide an active agent to a subject in the gastrointestinal tract at a point after the mouth. As such, the compositions and methods of the present method can be considered comestible as defined herein.

In another embodiment, the non-resilient art-known granules and the resilient granules of the present invention may both comprise a pharmaceutically active agent. In some aspects, the active agent can be the same or in some aspects, the active agent can be different in each granule type. Further, the dosage of the active agent in each case may be the same or different. For example, where the active agent is the same in both resilient and non-resilient granules, the non-resilient granules may be coated to provide modified release for the active agent while the resilient granules of the invention provide an immediate delivery of the active agent. In another embodiment, different active agents may be present a single granule or individually present in individual granules. Also, a combination of these granules may be present as immediate release, controlled release, or mixtures thereof. A variety of dosage and release characteristics can be obtained by following the concepts and examples presented herein.

The unique characteristics of the present granules permit incorporation of a wide variety of active ingredients, regardless of their aqueous solubility or particle size. For example, highly water soluble, sparingly water soluble, and water insoluble actives can be employed. Particle sizes of the actives could range from about 50 nm to about 500 uM. Thus, nanoparticles and microparticles can be used to make the present resilient, self-adhering granules.

The active agents for example can include, but are not limited to, analgesics, anti-inflammatory agents, anthelmintics, anti-arrhythmic agents, antibiotics (including penicillins), anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives (hypnotics and neuroleptics), astringents, beta-adrenoceptor blocking agents, blood products and substitutes, cardiac inotropic agents, contrast media, corticosteroids, cough suppressants (expectorants and mucolytics), diagnostic agents, diagnostic imaging agents, diuretics, dopaminergics (antiparkinsonian agents), haemostatics, immunological agents, lipid regulating agents, muscle relaxants, parasympathomimetics, parathyroid calcitonin and biphosphonates, prostaglandins, radio-pharmaceuticals, sex hormones (including steroids), anti-allergic agents, stimulants and anoretics, sympathomimetics, thyroid agents, vasodilators, and xanthines.

Other active agents include, without limitation, antitussives; decongestants; alkaloids; laxatives; antacids; ion exchange resins; anti-cholesterolemics; antipyretics; analgesics including acetaminophen, aspirin, non-asteroidal anti-inflammatory drugs (“NSAID”) and opioids; appetite suppressants; expectorants; anti-anxiety agents; anti-ulcer agents; coronary dilators; cerebral dilators; peripheral vasodilators; anti-infectives; psycho-tropics; antimanics; stimulants; gastrointestinal agents; sedatives; anti-diarrheal preparations; anti-anginal drugs; vasodilators; vasoconstrictors; migraine treatments; tranquilizers; anti-psychotics; antitumor drugs; antithrombotic drugs; hypnotics; anti-emetics; anti-nausants; anti-convulsants; neuromuscular drugs; hyper- and hypoglycemic spasmodics; uterine relaxants; antiobesity drugs; anabolic drugs; erythropoetic drugs; antiasthmatics; mucolytics; anti-uricemic drugs; and mixtures thereof. In one embodiment, the active agent is selected from the group consisting of: an analgesic, an antibiotic, a lipid regulating agent, an antihistamine, an antineoplastic agent, and an antiviral agent.

Additionally, nutritional active materials may be delivered using the resilient, self adhering granules of the present invention and include, without limitation, calcium-containing materials such as calcium carbonate, stannol esters, hydroxycitric acid, vitamins, minerals, herbals, spices, and mixtures thereof. Examples of vitamins that are available as active ingredients include, without limitation, vitamin A (retinol), vitamin D (cholecalciferol), vitamin E group, vitamin K group (phylloquinones and menaquinones), thiamine, riboflavin, niacin, folic acid, cobalamins, biotin, vitamin C (ascorbic acid), and mixtures thereof. The amount of vitamin or vitamins present in the final product of the present inventive subject matter is dependent on the particular vitamin and is generally the United States' Department of Agriculture Recommended Daily Allowances (USRDA) for that vitamin. For example, if vitamin C is the active ingredient, the amount of vitamin C in the encapsulated product would be 60 milligrams, which is the USRDA of vitamin C for adults. In one embodiment, the nutritional material can be zinc or calcium.

Pharmaceutical compositions according to the invention may also comprise one or more binding agents, filling agents, lubricating agents, suspending agents, emulsifiers, sweeteners, flavoring agents, preservatives, buffers, wetting agents, disintegrants, effervescent agents, and other excipients. Such excipients are well-known in the art.

Resilient, self-adhering granules of the invention can be prepared by a variety of granulation techniques known in the art. For example, granulation can be accomplished by granulating in a fluidized bed and admixture comprising an active agent and one or more pharmaceutically acceptable water-soluble or water-dispersible excipients, to form a granule. Alternatively, granulation may be performed by using high shear granulation.

The resilient self-adhering granules of the invention can be formulated into several known oral dosage forms. For example, tablets can be prepared by pharmaceutical compression or molding techniques known in the art. In addition, powders for administration can be prepared from the granules of the present invention or directly as granulated powders by any method known in the art. For example, such methods include, but are not limited to, milling, fluid bed granulation, dry granulation, direct compression, spheronization, spray congealing, and spray-dying. Detailed descriptions of tabletting methods are provided in Remington: The Science and Practice of Pharmacy, 19th ed Vol. 11 (1995) (Mack Publishing Co., Pennsylvania); and Remington's Pharmaceutical Sciences, Chapter 89, pp. 1633-1658 (Mach Publishing Company, 1990), both of which are specifically incorporated by reference.

In one aspect, an active agent, and at least one pharmaceutically acceptable water-soluble or water-dispersible excipient, and, optionally, other excipients are mixed to form a blend which is then directly compressed into tablets. For example, an active agent can be blended with tablet excipients using a V-blender or high-shear mixer to produce free-flowing compressible granules, which may be sieved for size uniformity. This may be followed by compression of the powder-granules using, for example, an automated press, or a single station press, or a high-speed tablet press.

The tablets may be further coated or uncoated. If coated, they may be sugar-coated or film-coated (to cover objectionable tastes or odors and to protect against oxidation) or coated with a release-modifying coating material such as an enteric coating. In accordance with coatings of art-known non-resilient granules tablets and caplets, a variety of coating materials and techniques are available to coat the tablets and caplets and granules of the present invention and to effect a variety of release patterns.

The present invention also provides methods to produce resilient, self-adhering granules. In one aspect, the method may comprise of the following steps:
a) heating mixed fats and emulsifiers to about 120 to about 220 F to obtain liquid consistency;
b) adding carbohydrates to said liquid of step a), optionally heating to about 120 to about 180 F;
c) adding active agent, and optionally excipients selected from the group consisting of sweeteners, swelling agents, flavoring agents, binders, disintegrants, bulking agents, and mixtures thereof;
d) adding a binding agent and optionally blending;
e) lowering temperature of the composition formed in step d) to a temperature below room temperature; and
f) subjecting said composition of step e) to size-reduction methods selected from the group consisting of: milling, shearing, sieving, or a combination thereof to obtain substantially uniform sized compressible resilient granules.

In some aspects, the method further comprises adding lubricants and other tabletting ingredients such as flavorants, sweeteners and colorants, and optionally blending and compressing into an oral dosage form.

In another aspect, the method comprises using cooling aids such as dry ice to prevent stickiness prior to the milling step. In another aspect, the method comprises using a lubricant such as silicon dioxide in an amount greater than about 1.5% w/w of the composition. In another aspect, the lubricant is greater than about 2% w/w of the composition. In another aspect, the lubricant is greater than about 2.5% w/w of the composition. In another aspect, the lubricant is greater than about 3% w/w of the composition.

The present invention also provides methods of administering an active agent to a subject including providing the active agent in an oral dosage form, said oral dosage form can comprise resilient self-adhering granules as previously discussed, administering the oral dosage form to the subject's oral cavity, such that the majority of the active agent can be released in the gastrointestinal tract at a point after the mouth.

In one embodiment, the tablet can be administered by breaking the tablet into two portions approximately defined by a scoring, where the breaking results in substantially no material loss. Additionally, the tablet can be reformed with substantially no material loss. In one embodiment, the oral dosage form can be characterized as not a gum or chew formulation.

### EXAMPLES

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

### Example 1

### Zinc Acetate and Zinc Gluconate Comprising Resilient Granules

In this Example, Zinc Acetate and Zinc Gluconate comprising resilient granules were prepared. The combined amount of elemental zinc was approximately 37 mg in the final compressed product. The composition had the following ingredients: mono- and di-glyceride emulsifiers (Durem 117) (60 mg); Panalite 90 DK (maltitol syrup) (30 mg); polyethylene glycol 3350 (40 mg); partially hydrogenated soy bean oil and cotton seed oil (Kaomel) (10 mg); acetylated mono- and di-glycerides (Myvacet) (50 mg); maltodextrin (Maltrin M-180) (171 mg); maltitol syrup (Lycasin HDS) (200 mg); methylcellulose (Methocel K100) (5 mg); granulated sugar (285 mg); sweeteners and colorants and flavoring aids (about 35 mg).

The fats and emulsifying agents were mixed together in a mixer and heated till the ingredients were melted. Maltitol syrup was then added to the molten fat and was mixed thoroughly. To this was added the active ingredient and mixed further for about 10 minutes. The molten and mixed mass was then extruded in an extruder and the ropes were collected into trays. The ropes then were allowed to condition at room temperature for about 6-8 hrs. The dried roped material was then milled to desired particle size. During milling, dry ice was used as needed to avoid sticking of the material. The material was then cooled in a freezer or was used directly to lubrication and compression.

The resilient material was sifted through an appropriate mesh for the desired particle size. The sifted material was then lubricated in a mixer by using a lubricant or lubricant mixture. In this particular example, the lubricant was a mixture of magnesium stearate and talc comprising about 2.5% w/w of the final composition; however, other lubricants may be used. If needed, depending on the particular active ingredient, colloidal silicon dioxide may also be used in addition to the above mixture of lubricants. If used, colloidal silicon dioxide may comprise about 3% w/w of the final composition. Sweeteners, flavors, and colorants as desired my then be added to the blending mixture. If used, sweeteners, flavors, and colorants may add up to about 5% w/w of the final composition. The mixer was run at 40 rpm and the mixing was continued for 10 minutes. The material was then frozen in a refrigerator for about 6 hrs prior to compression. The resilient granules were then compressed into tablets.

The uncoated tablets comprising resilient granules were then coated in a coating pan using a coating mixture comprising Crystalac C and talc. The coating composition amounted to about 5% w/w of the final composition.

### Example 2

### Zinc Acetate Comprising Compressible Resilient Granules

Example 1 was followed to make compositions comprising zinc acetate as the active agent. The composition comprises zinc acetate: 5.6% w/w.

### Example 3

### Sumatriptan Comprising Compressible Resilient Granules

The composition comprised 20.1% w/w of sumatriptan, 24.5% w/w of mixed fats and emulsifiers, 17% w/w of maltodextrin, 23% w/w of maltitol syrup, with the remaining to include flavors, sweeteners, swelling agents, coloring agents, and lubricating agents. The sumatriptan employed is an encapsulated taste-masked formulation prepared by art-known processes such as resin-complexation, complex coacervation, polymer-coating, and wax-fat coating. The sumatriptan formulation provides 100 mg per dosage form.

The process for granule preparation was as follows. Mixed fats and emulsifiers were heated to 180-190 F to obtain liquid consistency. Maltitol syrup was added to this liquid mixture and heating continued to about 150 F. The mixture was then transferred to a Sigma mixer using front blade at 60 RPM for about 1-3 minutes. The taste-masked sumatriptan, sweeteners, and swelling agents were added to this mixture, and blending continued for a few more minutes at 60 RPM. Maltodextrin and flavors were added to this mixture, and blending continued for further few minutes. Alternatively, sumatriptan may be added at this stage. The mixture was then cooled. This was followed by size reduction using multi-mill with knife blade forward and screened to obtain uniform granule size using a size 12 screen. Lubricants and other tabletting ingredients including a glidant were added to these granules and the mixture was blended for a few minutes. The mixture was then transferred to a tabletting machine for compression into tablets of desired shape and size.

### Example 4

### Sumatriptan Comprising Compressible Resilient Granules

The process described in Example 3 was followed with the exception that sumatriptan was added at the lubrication stage.

### Example 5

### Uncoated Active Ingredient Comprising Compressible Resilient Granules

The Example of either 1 or 2 is followed except that the active agent is uncoated drug and the drug is loratadine. The loratadine is present from about 5 mg to 20 mg. The remaining ingredients of the composition are adjusted accordingly to take into consideration the total weight percentages to substantially remain the same as previously described.

### Example 6

### Compressible Resilient Granules Mixed with Coated Modified Release Granules

The Example of either 1 or 2 is followed except that the resilient compressible granules are made with no active agent. Once the compressible resilient granules are made, these are mixed with granules or powder comprising active ingredients that are coated with a delayed release coating. Olanzapine composition that is coated with a delayed release coating is used as an example to deliver 20 mg olanzapine per dosage form. Preparation of such delayed release coated olanzapine is known in the art. Granules of delayed release coated olanzapine are then mixed with compressible resilient granules at the lubrication stage into the final blend and the mixture is then compressed into a tablet as described above. The dosage form delivers modified release olanzapine.

### Example 7

### Compressible Resilient Granules Mixed with Coated Modified Release Granules

The Example of 6 is followed except that the resilient compressible granules are made with olanzapine that is not coated. These compressible granules are then mixed with granules of delayed release coated olanzapine at the lubrication stage into the final blend and the mixture is then compressed into a tablet as described above. The dosage form delivers an initial immediate release dose of olanzapine followed by delayed release olanzapine to produce therapeutic activity over a longer duration, preferably for 6-8 hrs or even longer, if desired.

### Example 8

### Compressible Resilient Granules Mixed with Coated Modified Release Granules of Two Different Actives

The Example of either 1 or 2 is followed except that the resilient compressible granules are made with the active agent comprising amlodipine. Amlodipine besylate can be used as an example to deliver 5 mg. Amlodipine may be encapsulated for taste-masking purposes if needed. Separately, compressible resilient granules comprising atorvastatin calcium are made. The atorvastatin granules are made to deliver 10, 20, 40, or 80 mg per dosage form and optionally are coated for taste-masking purposes as needed. These two compressible resilient granules are then mixed, at the lubrication stage into the final blend and the mixture is then compressed into a tablet as described above. The dosage form delivers combination of both amlodipine and atorvastatin.

### Example 9

### Compressible Resilient Granules Mixed with Coated Modified Release Granules of Two Different Actives and Different Release Rates

The Example of 8 is followed except that either amlodipine or atorvastatin may be prepared as delayed-release granules while the other drug is prepared as compressible resilient granules. The two types of granules are then mixed, at the lubrication stage into the final blend and the mixture is then compressed into a tablet as described above. The dosage form delivers an immediate release of either amlodipine or atorvastatin while providing modified release delivery of the other active.

Of course, it is to be understood that the above-described arrangements are only illustrative of the application of the principles of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. A resilient self-adhering granule, which includes a combination of a polysaccharide in an amount of about 10 wt % to about 30 wt %; and a binder having a viscosity from about 5,000 mPa·s to about 250,000 mPa·s in an amount of from about 10 wt % to about 35 wt %, that is capable of low-pressure, reversible agglomeration.

2. The granule of claim 1, further comprising a sugar alcohol in an amount of about 15 wt % to about 35 wt %, wherein the sugar alcohol is selected from the group consisting of arabitol, erythritol, hydrogenated starch hydrolysates, isomalt, lactitol, maltitol, mannitol, sorbitol, xylitol, galactitol, inositol, ribitol, dithioerythritol, dithiothreitol, glycerol, derivatives thereof, and mixtures thereof.

3. The granule of claim 2, wherein the sugar alcohol is maltitol.

4. The granule of claim 1, wherein the polysaccharide is selected from the group consisting of simple sugars, complex sugars, fibers, starches, pectins, dextrans, dextrins, natural gums, synthetic gums, mucilages, derivatives thereof, components thereof, and mixtures thereof.

5. The granule of claim 4, wherein the polysaccharide is a dextrin.

6. The granule of claim 5, wherein the dextrin is maltodextrin.

7. The granule of claim 1, further comprising an active agent selected from the group consisting of analgesics; anti-inflammatory agents; anthelmintics; anti-arrhythmic agents; antibiotics including penicillins; anticoagulants; antidepressants; antidiabetic agents; antiepileptics; antihistamines; antihypertensive agents; antimuscarinic agents; antimycobacterial agents; antineoplastic agents; immunosuppressants; antithyroid agents; antiviral agents; anxiolytic sedatives including hypnotics and neuroleptics; astringents; beta-adrenoceptor blocking agents; blood products and substitutes; cardiac inotropic agents; contrast media; corticosteroids; cough suppressants including expectorants and mucolytics; diagnostic agents; diagnostic imaging agents; diuretics; dopaminergics including antiparkinsonian agents; haemostatics; immunological agents; lipid regulating agents; muscle relaxants; parasympathomimetics; parathyroid calcitonin and biphosphonates; prostaglandins; radio-pharmaceuticals; sex hormones including steroids; anti-allergic agents; stimulants and anoretics; sympathomimetics; thyroid agents; vasodilators; xanthines; antitussives; decongestants; alkaloids; laxatives; antacids; ion exchange resins; anti-cholesterolemics; antipyretics; analgesics including acetaminophen, aspirin, non-asteroidal anti-inflammatory drugs (“NSAID”) and opioids; appetite suppressants; expectorants; anti-anxiety agents; anti-ulcer agents; coronary dilators; cerebral dilators; peripheral vasodilators; anti-infectives; psycho-tropics; antimanics; stimulants; gastrointestinal agents; sedatives; anti-diarrheal preparations; anti-anginal drugs; vasodilators; vasoconstrictors; migraine treatments; tranquilizers; anti-psychotics; antitumor drugs; antithrombotic drugs; hypnotics; anti-emetics; anti-nausants; anti-convulsants; neuromuscular drugs; hyper- and hypoglycemic spasmodics; uterine relaxants; antiobesity drugs; anabolic drugs; erythropoetic drugs; antiasthmatics; mucolytics; anti-uricemic drugs; and mixtures thereof.

8. The granule of claim 7, wherein the active agent is selected from the group consisting of: an analgesic, an antibiotic, a lipid regulating agent, an antihistamine, an antineoplastic agent, and an antiviral agent.

9. The granule, of claim 7, wherein the active agent is homogenous throughout the granule.

10. The granule of claim 7, wherein the active agent is coated on the surface of the granule.

11. The granule of claim 7, wherein the active agent is configured for controlled release.

12. The granule of claim 7, wherein the active agent is configured for immediate release.

13. The granule of claim 1, further comprising an additional additive selected from the group consisting of filling agents, lubricating agents, suspending agents, sweeteners, flavoring agents, preservatives, buffers, wetting agents, disintegrants, effervescent agents, and mixtures thereof.

14. The granule of claim 1, wherein the binder is selected from the group consisting of syrups, emulsifiers, fats, waxes, gums, plasticizers, and mixtures thereof.

15. The granule of claim 14, wherein the binder is selected from the group consisting of maltitol syrups; acetylated mono-, di-, or triglycerides; polyethyleneglycol esters; bees wax, carnuba wax; spermaceti; mineral oils; paraffins; microcrystalline waxes; polyethylene wax; gum Arabica; gum tragacanth; gum acacia; fiber gums; and mixtures thereof.

16. The granule of claim 15, wherein the binder is maltitol syrup.

17. The granule of claim 15, wherein the binder has a viscosity of at least about 10,000 mPa·s.

18. The granule of claim 1, further comprising a nutritional supplement selected from the group consisting of calcium-containing materials, stannol esters, hydroxycitric acid, vitamins, minerals, herbals, spices and mixtures thereof.

19. The granule of claim 18, wherein the vitamin is selected from the group consisting of, vitamin A, vitamin D, vitamin E group, vitamin K group including phylloquinones and menaquinones, thiamine, riboflavin, niacin, folic acid, cobalamins, biotin, vitamin C, and mixtures thereof.

20. The granule of claim 18, wherein the nutritional supplement is a calcium-containing material, a zinc-containing material or vitamin C.

21. The granule of claim 1, wherein the granules agglomerate at a pressure of about 6500 kilonewtons/m².

22. The granule of claim 21, wherein the granules withstand compression pressures of about 40 kilonewtons without losing their resiliency.

23. The granule of claim 1, wherein the granules withstand compression pressures of about 50 kilonewtons without losing their resiliency.

24. The granule of claim 1, wherein the granule is produced by mixing and heating the binder, polysaccharide, and sugar alcohol in a mixer to form a reaction mixture, mixing for about 10 minutes, extruding the reaction mixture, cooling to room temperature for about 6-8 hours, milling to a particular granule size, and cooling in a freezer.

25. The granule of claim 1, wherein the granule is lubricated with a lubricant, and further modified with sweeteners, flavors and/or colorants.

26. An oral dosage composition, comprising a plurality of resilient granules as recited in claim 1, wherein the granules are combined into an oral dosage composition.

27. The composition of claim 26, wherein the plurality of resilient granules further comprises active agents homogenous throughout or coated thereon.

28. The composition of claim 27, wherein the granules contain a plurality of active agents present in a single granule or individually present in individual granules.

29. The composition of claim 28, wherein the granules are configured for immediate release, controlled release, or mixtures thereof.

30. The composition of claim 29, further comprising granules without active agents.

31. The composition of claim 26, wherein the granules are admixed with non-resilient granules having an active agent.

32. The composition of claim 26, wherein the oral dosage composition is a tablet or a semi-solid oral composition.

33. The composition of claim 32, wherein the tablet has been scored at least once.

34. The composition of claim 33, wherein the tablet is broke into two portions approximately defined by the scoring, and said breaking results in substantially no material loss.

35. The composition of claim 34, wherein the two portions are reformed with substantially no material loss.

36. Method of administering an active agent to a subject comprising,
a) providing the active agent in an oral dosage form, said oral dosage form comprises resilient granules as recited in claim 1,
b) administering the oral dosage form to the subject's oral cavity, wherein the majority of the active agent is released in the gastrointestinal tract at a point after the mouth.

37. The method of claim 36, wherein the active agent is present in the resilient granules.

38. The method of claim 36, wherein the active agent is present in a non-resilient granule form.

39. The method of claim 36, wherein the oral dosage form is a tablet.

40. The method of claim 39, wherein the tablet is scored at least once.

41. The method of claim 40, the tablet is administered by breaking the tablet into two portions approximately defined by the scoring, and said breaking results in substantially no material loss.

42. The method of claim 36, wherein the oral dosage form is not a chewing gum.

43. The composition of claim 7, wherein the lubricating agent comprises greater than about 2% w/w of the composition

44. The composition of claim 43, wherein the lubricating agent is silicon dioxide.
